(19) United States
(12) Patent Application Publication
(10) Pub. No.: US 2021/0404905 A1
(43) Pub. Date: Dec. 30, 2021

(54) SYSTEM FOR REAL-TIME DETECTING LEAKAGE OF UNDERGROUND VOLATILE COMPOUND

(71) Applicants: Sung Kook CHO, Pyeongtaek-si, Gyeonggi-do (KR); HYORIM Co., Ltd., Seongnam-si (KR)

(72) Inventors: Sung Kook CHO, Pyeongtaek-si (KR); Jung Su AN, Incheon (KR); Cheol Soon CHOI, Suwon-si (KR); Sung Heui CHO, Pohang-si (KR); Myeong Gwang OH, Yongin-si (KR); Sang hwan LEE, Gangneung-si (KR)

(73) Assignees: HYORIM CO., Ltd.; Sung Kook Cho

(21) Appl. No.: 17/475,499

(22) Filed: Sep. 15, 2021

Related U.S. Application Data
(63) Continuation of application No. PCT/KR2019/012116, filed on Sep. 19, 2019.

(30) Foreign Application Priority Data
Mar. 21, 2019 (KR) 10-2019-0032227

(51) Int. Cl. G01M 3/18; H04Q 9/00; G06F 3/14

(52) U.S. Cl. CPC G01M 3/186; H04Q 2209/40; G06F 3/14; H04Q 9/00

(57) ABSTRACT

The present invention relates to a real-time underground volatile organic compound leakage detection system and, more specifically, to a real-time underground volatile organic compound leakage detection system for minimizing contamination and allowing a rapid response by detecting beforehand in real time the spread of contamination in the soil and groundwater from environmental accidents due to leaks of oil, overflow oil and the like from the ground and underground storage tanks of a facility which is to be monitored for oils and harmful chemical substances comprising volatile organic compounds (VOCs). The system comprises: a monitoring well, provided in the periphery of the facility to be monitored, for collecting volatile organic compounds (VOCs) leaked from the facility to be monitored; a gas sensor module provided inside the monitoring well and for detecting the volatile organic compounds (VOCs) and measuring the concentration; a communication module for wirelessly transmitting detection and concentration sensor data measured by means of the gas sensor module; and a control center server for displaying the detection and concentration sensor data received from the communication module, and outputting a monitoring screen.

### REFERENCE TO RELATED APPLICATIONS

This is a continuation of International Patent Application PCT/KR2019/012116 filed on Sep. 19, 2019, which designates the United States and claims priority of Korean Patent Application No. 10-2019-0032227 filed on Mar. 21, 2019, the entire contents of which are incorporated herein by reference.

### FIELD OF THE INVENTION

The present invention relates to a real-time underground volatile-organic-compound leakage detection system, and more particularly to a real-time underground volatile-organic-compound leakage detection system for minimizing contamination and allowing a rapid response by preemptively detecting, in real time, the spread of contamination in the soil and groundwater from environmental accidents due to leakage of oil, overflowing oil and the like from surface and underground storage tanks of a facility which is to be monitored for oils and harmful chemicals including volatile organic compounds (VOCs), the system including a monitoring well provided near the facility to be monitored and configured to collect volatile organic compounds (VOCs) leaking from the facility to be monitored, a gas sensor module provided inside the monitoring well and configured to detect the volatile organic compounds (VOCs) and measure the concentration thereof, a communication module configured to wirelessly transmit detection and concentration sensor data measured using the gas sensor module, and a control center server configured to display the detection and concentration sensor data received from the communication module and output a monitoring screen.

### BACKGROUND OF THE INVENTION

In general, oil or chemicals stored in the surface or underground storage tanks of facilities that are buried underground or provided on the ground and are to be monitored for harmful chemicals including volatile organic compounds (VOCs), etc. and soil contaminants, are regarded as potential soil and groundwater contaminants that may contaminate the underground environment.

For example, leakage of oil or harmful chemicals from oil storage tanks or harmful chemical storage tanks and pipes of gas stations buried underground damages the underground environment and causes direct contamination of soil or groundwater, and moreover, soil and groundwater may be contaminated due to leakage caused by corrosion, aging, or breakage of underground facilities.

Typically, when an oil or harmful chemical starts leaking underground, the leakage may only be recognized by measuring the amount of oil that is lost due thereto or directly detecting such a contaminant. For example, when oil leaks from underground storage tanks or pipes at gas stations, the leakage may be detected by measuring a decrease in the amount of oil stored in the storage tanks, or leaked oil that leaks and seeps into the soil may be detected using a detection device buried underground.

However, there is often the case in which the oil leakage cannot be reliably detected using such a detection device. For example, in the case in which oil leaks in a small amount from a storage tank over a long period of time, the extent of soil contamination accumulates and becomes progressively more serious, but the amount of oil that is lost from the storage tank is relatively insignificant, so it is not easy to accurately measure such an amount.

Accordingly, various techniques capable of detecting contaminant leakage in real time have been developed. In this regard, Korean Patent No. 10-0467835 (Registration date: Jan. 13, 2005) discloses a contaminant leakage detection system using a grid-net electrical resistance measurement method, including: grid-type wires arranged within the ground at a location at which there is concern about leakage of contaminants; an electrical resistance measurement sensor including two electrodes connected to each of the intersections of the grid-type wires and a support configured to constantly fix the two electrodes; a current supply device configured to supply current to the grid-type wires; and a potential difference measurement device configured to measure the potential difference of the current flowing through the sensor, in which the point of decrease in the electrical resistance obtained through the measurement of the potential difference is determined to be a contaminant leakage point.

In addition, Korean Utility Model No. 20-0453056 (Registration date: Mar. 29, 2011) discloses a volatile-organic-compound detector for detecting volatile organic compounds contained in ambient air, including: a gas inlet 10 into which predetermined ambient air containing volatile organic compounds is introduced from the outside; an air inlet 20 into which predetermined clean air not containing volatile organic compounds is introduced from a separate tank or from the outside; a valve 30 configured to open and close the gas inlet 10 and the air inlet 20; a sensor array 40 configured to detect the volatile organic compounds contained in the ambient air introduced by opening and closing the valve 30; a circulation pump 50 configured to forcibly circulate the ambient air or clean air introduced into the sensor array 40 in a canister 41; a discharge port 60 provided downstream of the circulation pump 50 and configured to discharge the ambient air or clean air to the outside; a sensor amplifier 70 configured to amplify a data signal obtained from the sensor array 40; a sensor CPU 80 configured to calculate a reference voltage based on the data signal amplified by the sensor amplifier 70; a main controller 90 configured to receive the data signal calculated by the sensor CPU 80, control the valve 30 and the circulation pump 50, and control a screen display or an audio output signal based on the calculated data value; an LCD unit 100 controlled by the main controller 90 and configured to display analysis data of the ambient air in the form of a text, a diagram, or a graph; and an audio output unit 110 controlled by the main controller 90 and configured to output an audio when the measured value exceeds the preset reference value, in which the sensor array 40 includes the straight-type canister 41 made of stainless steel so that the ambient air or clean air flows smoothly, and in which a temperature sensor, a humidity sensor, and seven olfactory sensors 42 are paired in left-and-right or up-and-down and arranged parallel to each other on the outer surface of the canister 41.

In addition, Korean Patent No. 10-1692926 (Registration date: Dec. 29, 2016) discloses a harmful chemical leakage detection and response system for detecting and responding to harmful chemicals leaking from a vehicle carrying harmful chemicals, including: at least one sensor unit installed on a vehicle carrying harmful chemicals and configured to detect the presence and concentration of harmful chemicals within a predetermined range from the installation area; and a server configured to receive the detection information from the sensor unit and analyze the leakage of harmful chemicals in the area in which the corresponding sensor unit is installed, the sensor unit including a GPS module configured to obtain location information of the corresponding sensor unit and a communication module configured to communicate with the server. Here, when the concentration of harmful chemicals is detected above the preset numerical value, the location information of the sensor unit obtained from the GPS module and the harmful chemical leakage information thereof are transmitted to the server through the communication module. In the harmful chemical leakage detection and response system, the server includes a communication unit configured to communicate with the communication module and send a request for transmission of surrounding geographic information and weather information for the location information of the sensor unit to a related organization, a first processing unit configured to perform harmful chemical diffusion modeling by analyzing and processing the surrounding geographic information and weather information transmitted from the related organization and the harmful chemical leakage information transmitted from the sensor unit and calculating the concentration of emitted harmful chemicals, and a second processing unit configured to plot the harmful chemical diffusion modeling result obtained from the first processing unit as an isoconcentration curve on a map and output the plot result on a display, and the communication unit transmits the harmful chemical leakage information including the location information as well as the harmful chemical diffusion modeling result obtained from the first and second processing units to the corresponding organization.

In addition, Korean Patent No. 10-1767258 (Registration date: Aug. 4, 2017) discloses a chemical leakage alarm system including: a leakage detection device including a plurality of chemical leakage detection sensors configured to detect a chemical leak; a signal conversion device configured to receive a leakage detection signal for chemical leakage from a specific chemical leakage detection sensor of the leakage detection device and convert the received leakage detection signal into a standard signal; a periodic inspection device configured to generate and provide facility status information about a chemical handling facility by checking the status of the chemical handling facility; and a chemical management device configured to determine whether there is a leak in response to the standard signal received from the signal conversion device, search for and provide an accident response manual for each chemical, and manage the status of the chemical handling facility based on the facility status information received from the periodic inspection device, the signal conversion device including: a first communication unit configured to receive the leakage detection signal or provide leakage alarm information; a standard signal converter configured to convert the received leakage detection signal into a standard digital signal; a facility information DB configured to store chemical handling facility information for each chemical leakage detection sensor; and a leakage alarm information generator configured to generate leakage alarm information by extracting chemical handling facility information corresponding to the specific chemical leakage sensor from the facility information DB and then combining the standard signal and the extracted chemical handling facility information, the chemical handling facility information including chemical information and handling facility information.

In addition, Korean Patent No. 10-1802734 (Registration date: Nov. 23, 2017) discloses a soil and groundwater contaminant detection system including: a saturated-zone-buried pipe 100 buried in an underground saturated zone (aquifer) in which contaminants are to be detected; an unsaturated-zone-buried pipe 200 buried in an underground unsaturated zone (soil layer) in which contaminants are to be detected; a set of gas detectors 300, 300′ of the same type embedded and buried in each of the saturated-zone-buried pipe 100 and the unsaturated-zone-buried pipe 200 and configured to extract and analyze contaminants from the saturated zone and the unsaturated zone using current; a ground power supply 400 configured to supply power required for operation of each of the gas detectors 300, 300′; a ground suction pump 500 connected to each of the gas detectors 300, 300′ via a suction pipe 510 and configured to provide a negative pressure within each of the gas detectors 300, 300′; and a ground console 600 installed on the ground and configured to receive and monitor the data analyzed by the gas detectors 300, 300′ through RS-485 communication with each of the gas detectors 300, 300′ while controlling the entire configuration. Here, each of the gas detectors 300 and 300′ includes: a gas detector body 310 that has a hollow inside, is open to the bottom, and includes a lower gas extraction unit 312 and an upper gas analysis unit 313 partitioned by a nonwoven-fabric filter 311 that enables gas to pass therethrough and foreign substances to be filtered, in which a plurality of through holes 312a, 312a′ is formed around the gas extraction unit 312 to communicate with the inside thereof so as to allow water and soil to pass therethrough, and in which the gas analysis unit 313 is connected to the suction pump 500 through the suction pipe 510 so as to allow movement of the gas extracted from the gas extraction unit 312 when applying a negative pressure to the gas analysis unit 313; a gas extractor 320 disposed in the gas extraction unit 312 and configured to receive power from the power supply 400 and extract the underground water into a gas; and a gas analyzer 330 disposed in the gas analysis unit 313 and configured to receive the gas extracted from the gas extraction unit 312, analyze the gas component, and transmit the analysis result to the console 600.

However, the grid-net electrical resistance measurement method disclosed in Korean Patent No. 10-0467835 determines the point of decrease in the electrical resistance value obtained through potential difference measurement as the contaminant leakage point, and may thus be applied only to leakage of liquid contaminants, making it possible to measure large amounts of liquid contaminants, but being incapable of measuring fine leaks.

In addition, the volatile-organic-compound detector disclosed in Korean Utility Model No. 20-0453056, the harmful chemical detection system disclosed in Korean Patent No. 10-1692926, and the chemical leakage alarm method and system disclosed in Korean Patent No. 10-1767258 are effective at detecting contaminant leaks on the ground, but is incapable of detecting underground contaminant leaks.

In contrast, the soil and groundwater contaminant detection system disclosed in Korean Patent No. 10-1802734 is capable of underground contaminant leakage detection, but the entire system is expensive and large due to the use of the gas analyzer that forcibly vaporizes the leaked contaminants and measures the vaporized gas, making it technically and economically difficult to commercialize the same.

### SUMMARY OF THE INVENTION

The present invention has been made keeping in mind the problems encountered in the related art, and an objective of the present invention is to provide a real-time underground volatile-organic-compound leakage detection system for minimizing contamination and allowing a rapid response by preemptively detecting, in real time, the spread of contamination in the soil and groundwater from environmental accidents due to leakage of oil, overflowing oil and the like from surface and underground storage tanks of a facility which is to be monitored for oils and harmful chemicals including volatile organic compounds (VOCs), the system including: a monitoring well provided near the facility to be monitored and configured to collect volatile organic compounds (VOCs) leaking from the facility to be monitored; a gas sensor module provided inside the monitoring well and configured to detect the volatile organic compounds (VOCs) and measure the concentration thereof; a communication module configured to wirelessly transmit detection and concentration sensor data measured using the gas sensor module; and a control center server configured to display the detection and concentration sensor data received from the communication module and output a monitoring screen.

In order to accomplish the above objective, the present invention provides a real-time underground volatile-organic-compound leakage detection system, including a monitoring well provided near a facility to be monitored for oils and harmful chemicals including volatile organic compounds (VOCs) and configured to collect volatile organic compounds (VOCs) leaking from the facility to be monitored, a gas sensor module provided inside the monitoring well and configured to detect the volatile organic compounds (VOCs) and measure the concentration thereof, a communication module configured to wirelessly transmit detection and concentration sensor data measured using the gas sensor module, and a control center server configured to display the detection and concentration sensor data received from the communication module and output a monitoring screen.

The gas sensor module may detect the volatile organic compounds (VOCs) depending on a change in an electrical resistance value when coming into contact with the volatile organic compounds (VOCs), and may measure the concentration thereof.

The gas sensor module may include an insulating substrate, a pair of thin-film electrodes formed on the insulating substrate, a conductive carbon powder configured to connect the pair of thin-film electrodes, and a permeable thin polymer film applied on the pair of thin-film electrodes and the conductive carbon powder.

The monitoring screen of the control center server may include a leakage detection map screen on which the detection and concentration sensor data measured using the gas sensor module of the monitoring well are displayed for each monitoring well on a map on which locations of monitoring wells provided near the facility to be monitored are marked.

The leakage detection map screen may separately output normal data indicating that the sensor data value displayed for each monitoring well is equal to or less than a preset allowable value, abnormality detection data indicating detection of an abnormality exceeding the normal data range, and leakage detection data indicating leakage detection exceeding the abnormality detection data range.

The monitoring screen of the control center server may further include a detection status screen on which the location of the facility to be monitored, the sensor data value for each well, the normal state, abnormality detection state, or leakage detection state for each well, the gas sensor module for each well, the communication module, the battery maintenance or replacement request, and the action status are displayed.

According to the present invention, a real-time underground volatile-organic-compound leakage detection system includes a monitoring well provided near a facility to be monitored for harmful chemicals including volatile organic compounds (VOCs), etc. and soil contaminants and configured to collect volatile organic compounds (VOCs) leaking from the facility to be monitored, a gas sensor module provided inside the monitoring well and configured to detect the volatile organic compounds (VOCs) and measure the concentration thereof, a communication module configured to wirelessly transmit detection and concentration sensor data measured using the gas sensor module, and a control center server configured to display the detection and concentration sensor data received from the communication module and output a monitoring screen. Thereby, harmful chemicals including volatile organic compounds (VOCs), etc., particularly volatile organic compound (VOC) gases, can be measured using the gas sensor, so even fine leaks can be detected, and moreover, the system can be reduced in size, thus effectively making it possible to realize size reduction and low cost from technical and economic aspects.

### BRIEF DESCRIPTION OF THE DRAWINGS

FIG. 1 illustrates the overall configuration of a leakage detection system according to the present invention;

FIG. 2 illustrates the structure of a gas sensor module according to the present invention;

FIG. 3 illustrates the sensor measurement principle of the gas sensor module according to the present invention;

FIG. 4 illustrates a leakage detection map on the monitoring screen of a control center server according to the present invention; and

FIG. 5 illustrates a detection status screen on the monitoring screen of the control center server according to the present invention.

### DETAILED DESCRIPTION OF THE INVENTION

The present invention pertains to a real-time underground volatile-organic-compound leakage detection system, including a monitoring well provided near a facility to be monitored for oils and harmful chemicals including volatile organic compounds (VOCs) and configured to collect volatile organic compounds (VOCs) leaking from the facility to be monitored, a gas sensor module provided inside the monitoring well and configured to detect the volatile organic compounds (VOCs) and measure the concentration thereof, a communication module configured to wirelessly transmit the detection and concentration sensor data measured using the gas sensor module, and a control center server configured to display the detection and concentration sensor data received from the communication module and output a monitoring screen.

The gas sensor module detects the volatile organic compounds (VOCs) depending on a change in the electrical resistance value when coming into contact with the volatile organic compounds (VOCs), and measures the concentration thereof.

The gas sensor module includes an insulating substrate, a pair of thin-film electrodes formed on the insulating substrate, a conductive carbon powder configured to connect the pair of thin-film electrodes, and a permeable thin polymer film applied on the pair of thin-film electrodes and the conductive carbon powder.

The monitoring screen of the control center server includes a leakage detection map screen on which the detection and concentration sensor data measured using the gas sensor module of the monitoring well are displayed for each monitoring well on a map on which the locations of monitoring wells provided near the facility to be monitored are marked.

The leakage detection map screen separately outputs normal data indicating that the sensor data value displayed for each monitoring well is equal to or less than a preset allowable value, abnormality detection data indicating detection of an abnormality exceeding the normal data range, and leakage detection data indicating leakage detection exceeding the abnormality detection data range.

The monitoring screen of the control center server further includes a detection status screen on which the location of the facility to be monitored, the sensor data value for each well, the normal state, abnormality detection state, or leakage detection state for each well, the gas sensor module for each well, the communication module, the battery maintenance or replacement request, and the action status are displayed.

Hereinafter, a detailed description will be given of embodiments of the present invention in conjunction with the accompanying drawings so that those of ordinary skill in the art can easily carry out the present invention. However, the present invention may be embodied in a variety of different forms, and is not limited to the embodiments and drawings described herein.

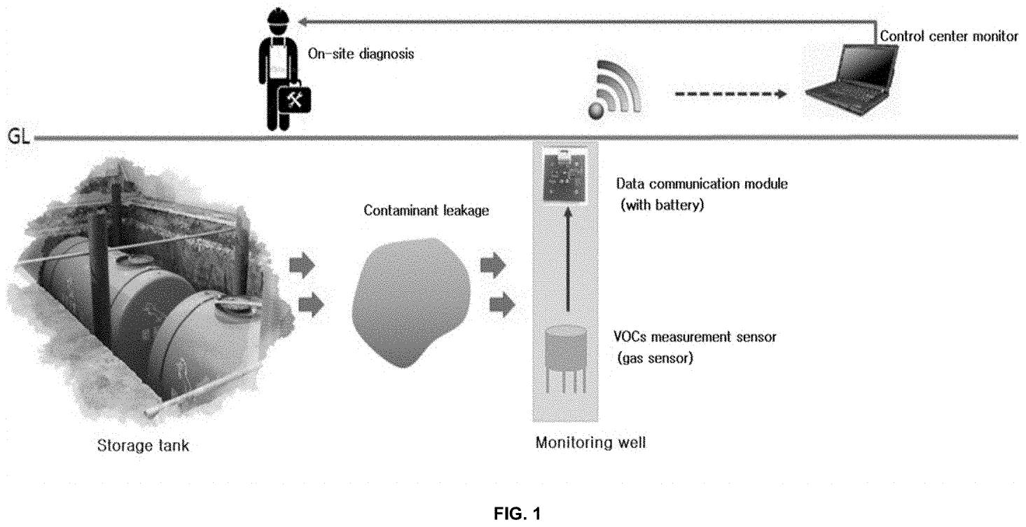

Specifically, with reference to FIG. 1, the real-time underground volatile-organic-compound leakage detection system according to the present invention includes a monitoring well provided near a facility to be monitored for oils and harmful chemicals including volatile organic compounds (VOCs) and configured to collect volatile organic compounds (VOCs) leaking from the facility to be monitored, a gas sensor module provided inside the monitoring well and configured to detect the volatile organic compounds (VOCs) and measure the concentration thereof, a communication module configured to wirelessly transmit detection and concentration sensor data measured using the gas sensor module, and a control center server configured to display the detection and concentration sensor data received from the communication module and output a monitoring screen.

Here, the gas sensor module is a gas sensor capable of detecting and sensing VOC gas, and conventional gas sensors include, for example, a complex sensor array for detecting a gas or volatile organic compound (VOC) disclosed in Korean Patent Application Publication No. 10-2016-0001369 (Laid-open date: Jan. 6, 2016), including a plurality of sensor arrays, each including a plurality of gas sensors configured to detect a gas or volatile organic compound (VOC), the sensor including a substrate, a lower electrode disposed on the substrate, a piezoelectric layer disposed on the lower electrode, an upper electrode disposed on the piezoelectric layer, and a gas-sensing layer disposed on the upper electrode, in which a plurality of sensors, in each of which a receptor (sensitive material) of a gas-sensing layer thereof is a carbon nanotube, and a plurality of sensors, in each of which a receptor is a polymer, are arranged on a single chip, and a complex gas detection sensor disclosed in Korean Patent Application Publication No. 10-2018-0135258 (Laid-open date: Dec. 20, 2018), including a support substrate and a gas detection unit cell disposed on the support substrate, the gas detection unit cell including an electrode layer including a plurality of electrodes and a gas detection construct having a porous structure and including a plurality of unit pattern layers laminated on the electrode layer. However, the gas-sensing layer has to be coated with a carbon nanotube (CNT)-polymer composite as the gas receptor, and the gas detection construct has to contain expensive metal such as tin oxide (SnOx), titanium oxide (TiOx), zinc oxide (ZnO), tungsten oxide (WO), etc. as the detection material, which is undesirable.

Hence, the gas sensor module according to the present invention is capable of detecting the volatile organic compounds (VOCs) depending on a change in the electrical resistance value when coming into contact with the volatile organic compounds (VOCs) and measuring the concentration thereof.

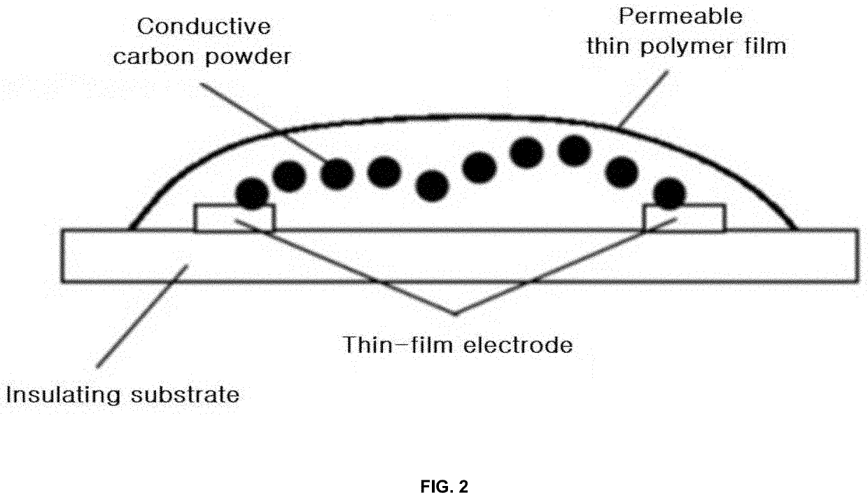

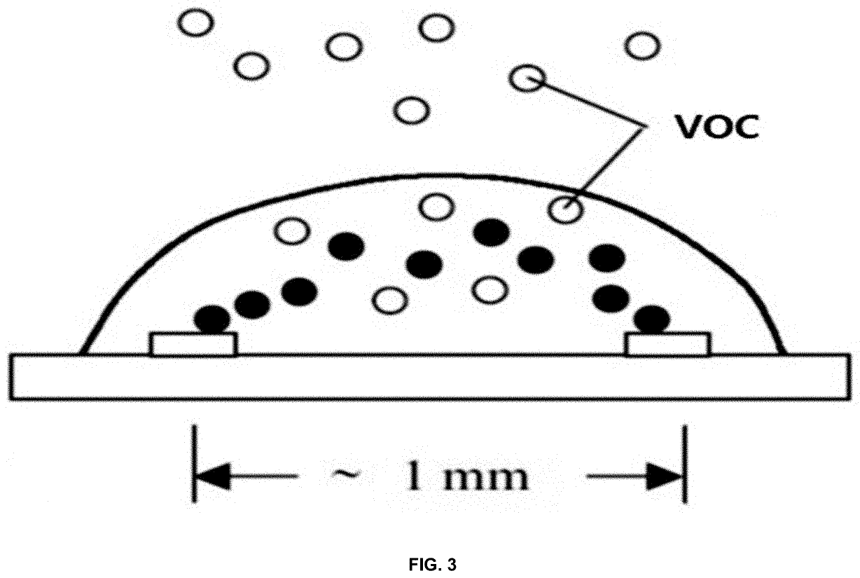

As illustrated in FIG. 2 to FIG. 3, the gas sensor module includes an insulating substrate, a pair of thin-film electrodes formed on the insulating substrate, a conductive carbon powder configured to connect the pair of thin-film electrodes, and a permeable thin polymer film applied on the pair of thin-film electrodes and the conductive carbon powder.

Here, the distance between the pair of thin-film electrodes that are spaced apart from each other may be designed to be a narrow width of 1 mm or less, and the gas sensor module of the present invention is preferably provided with a plurality of arrays including the pair of thin-film electrodes, which are spaced apart from each other at the fine width.

The process of detecting volatile organic compounds (VOCs) and measuring the concentration thereof using the gas sensor module is described below. When the gas sensor module is exposed to volatile organic compounds (VOCs), the volatile organic compounds (VOCs) pass through the permeable thin polymer film and are mixed with the conductive carbon powder. As such, the resistance value of the conductive carbon powder is changed, so the volatile organic compounds (VOCs) are detected depending on the changed value, and the concentration thereof is measured.

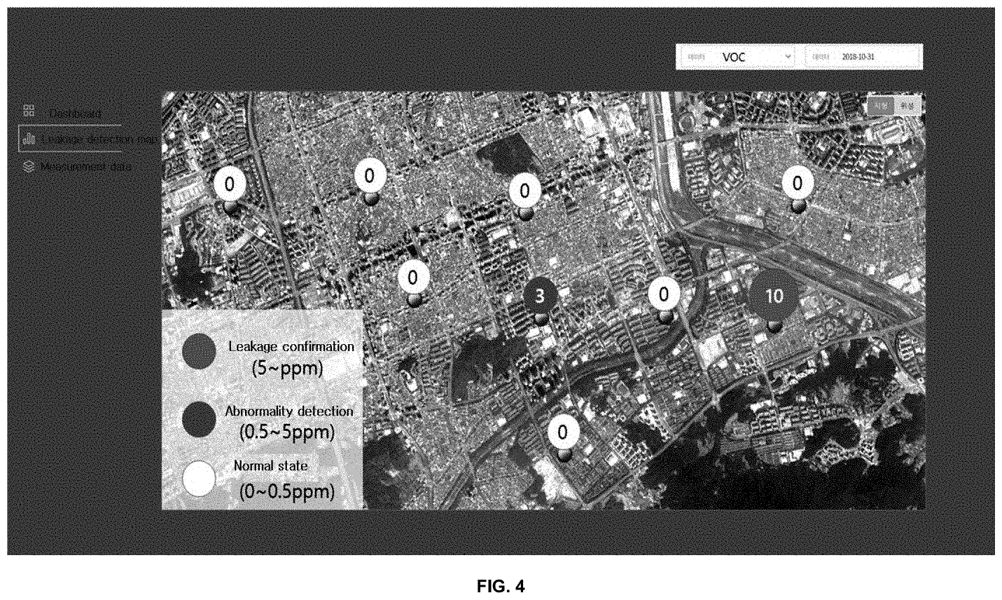

Meanwhile, as illustrated in FIG. 4, the monitoring screen of the control center server includes a leakage detection map screen on which the detection and concentration sensor data measured using the gas sensor module of the monitoring well are displayed for each monitoring well on a map on which the locations of monitoring wells provided near the facility to be monitored are marked.

Here, the leakage detection map screen may separately output normal data indicating that the sensor data value displayed for each monitoring well is equal to or less than a preset allowable value, abnormality detection data indicating detection of an abnormality exceeding the normal data range, and leakage detection data indicating leakage detection exceeding the abnormality detection data range.

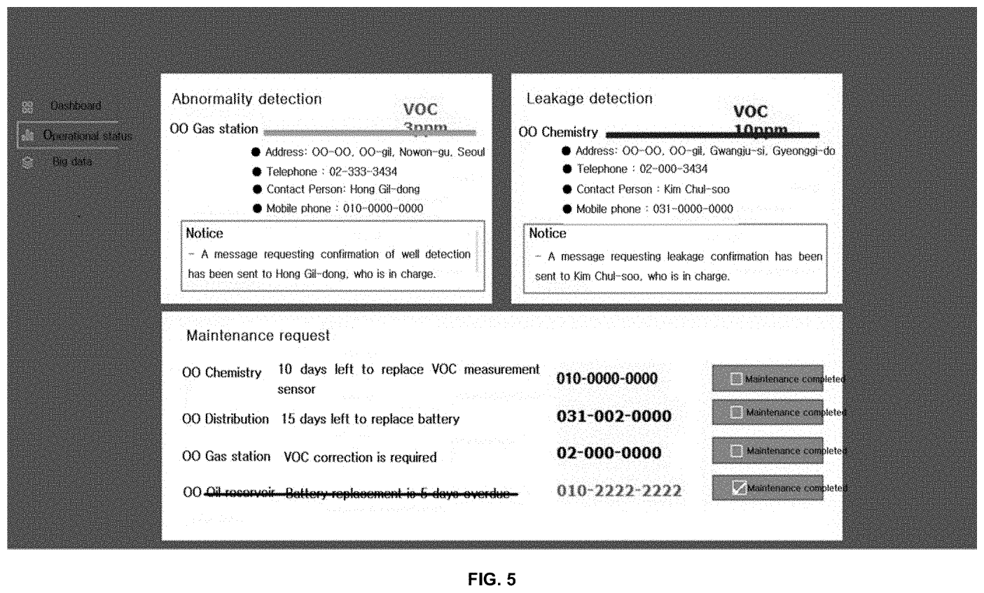

Moreover, as illustrated in FIG. 5, the monitoring screen of the control center server may further include a detection status screen on which the location of the facility to be monitored, the sensor data value for each well, the normal state, abnormality detection state, or leakage detection state for each well, the gas sensor module for each well, the communication module, the battery maintenance or replacement request, and the action status are displayed.

The above description is merely illustrative of the technical spirit of the present invention, and those skilled in the art to which the present invention belongs will appreciate that various modifications and variations are possible, without departing from the essential characteristics of the present invention. Therefore, the embodiments and drawings disclosed in the present invention are intended not to limit the technical spirit of the present invention but merely to explain the same, and the scope of the technical spirit of the present invention is not limited by these embodiments and drawings. The scope of the present invention is to be determined based on the accompanying claims, and all technical spirits within the scope equivalent thereto are to be construed as being included in the scope of the present invention.

According to the present invention, the real-time underground volatile-organic-compound leakage detection system includes a monitoring well provided near a facility to be monitored for harmful chemicals including volatile organic compounds (VOCs), etc. and soil contaminants and configured to collect volatile organic compounds (VOCs) leaking from the facility to be monitored, a gas sensor module provided inside the monitoring well and configured to detect the volatile organic compounds (VOCs) and measure the concentration thereof, a communication module configured to wirelessly transmit detection and concentration sensor data measured using the gas sensor module, and a control center server configured to display the detection and concentration sensor data received from the communication module and output a monitoring screen. Thereby, harmful chemicals including volatile organic compounds (VOCs), etc., particularly volatile organic compound (VOC) gases, can be measured using the gas sensor, so even fine leaks can be detected, and moreover, the system can be reduced in size, thus effectively making it possible to realize size reduction and low cost from technical and economic aspects, whereby the real-time underground volatile-organic-compound leakage detection system of the present invention is industrially applicable.

What is claimed is:

1. A real-time underground volatile-organic-compound leakage detection system, comprising:
a monitoring well provided near a facility to be monitored for oils and harmful chemicals including volatile organic compounds (VOCs) and configured to collect volatile organic compounds (VOCs) leaking from the facility to be monitored;
a gas sensor module provided inside the monitoring well and configured to detect the volatile organic compounds (VOCs) and measure a concentration thereof;
a communication module configured to wirelessly transmit detection and concentration sensor data measured using the gas sensor module; and
a control center server configured to display the detection and concentration sensor data received from the communication module and output a monitoring screen.

2. The real-time underground volatile-organic-compound leakage detection system according to claim 1, wherein the gas sensor module detects the volatile organic compounds (VOCs) depending on a change in an electrical resistance value when coming into contact with the volatile organic compounds (VOCs), and measures the concentration thereof.

3. The real-time underground volatile-organic-compound leakage detection system according to claim 1, wherein the gas sensor module comprises an insulating substrate, a pair of thin-film electrodes formed on the insulating substrate, a conductive carbon powder configured to connect the pair of thin-film electrodes, and a permeable thin polymer film applied on the pair of thin-film electrodes and the conductive carbon powder.

4. The real-time underground volatile-organic-compound leakage detection system according to claim 1, wherein the monitoring screen of the control center server comprises a leakage detection map screen on which the detection and concentration sensor data measured using the gas sensor module of the monitoring well are displayed for each monitoring well on a map on which locations of monitoring wells provided near the facility to be monitored are marked.

5. The real-time underground volatile-organic-compound leakage detection system according to claim 4, wherein the leakage detection map screen separately outputs normal data indicating that a sensor data value displayed for each monitoring well is equal to or less than a preset allowable value, abnormality detection data indicating detection of an abnormality exceeding a normal data range, and leakage detection data indicating leakage detection exceeding an abnormality detection data range.

6. The real-time underground volatile-organic-compound leakage detection system according to claim 4, wherein the monitoring screen of the control center server further comprises a detection status screen on which a location of the facility to be monitored, a sensor data value for each well, a normal state, an abnormality detection state, or a leakage detection state for each well, the gas sensor module for each well, the communication module, a battery maintenance or replacement request, and an action status are displayed.
